(19) United States
(12) Patent Application Publication
(10) Pub. No.: US 2009/0305389 A1
(43) Pub. Date: Dec. 10, 2009

(54) PERMEABLE MEMBRANES IN FILM PHOTOBIOREACTORS

(76) Inventors: Bryan Dennis WILLSON, Fort Collins, CO (US); Christopher Wayne TURNER, Windsor, CO (US); Guy Robert BABBITT, Fort Collins, CO (US); Peter Allan LETVIN, Fort Collins, CO (US); Sumith Ranil WICKRMASINGHE, Fort Collins, CO (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/481,418

(22) Filed: Jun. 9, 2009

Related U.S. Application Data
(60) Provisional application No. 61/059,863, filed on Jun. 9, 2008.

(51) Int. Cl. C12M 1/42; C12M 1/00; B29C 65/00

(52) U.S. Cl. 435/257.1; 435/292.1; 156/60

(57) ABSTRACT

Embodiments of the present invention include photobioreactors with membranes to introduce carbon dioxide into media contained within film photobioreactors. Such membranes can also be used to remove dissolved oxygen from the media. In some embodiments, one or more membrane tubes are welded into a plastic film photobioreactor to make a one-piece reactor. According to some embodiments of the present invention, algae is grown in a photobioreactor using pressure, gas composition, and surface area along with sparging to control the pH in the photobioreactor.

### CROSS-REFERENCE TO RELATED APPLICATIONS

This application claims the benefit of U.S. Provisional Patent Application Ser. No. 61/059,863, filed on Jun. 9, 2008, entitled, “Permeable Membranes in Film Photobioreactors,” which is incorporated herein by reference in its entirety for all purposes.

### TECHNICAL FIELD

Embodiments of the present invention relate generally to permeable membranes in photobioreactors, and more specifically to integration of porous and non-porous membranes and other porous materials into bioreactors to transfer gases to and from the media used to grow organisms.

### BACKGROUND

Producing biofuels, such as biodiesel, bioethanol, and/or biogasoline, from renewable energy sources provides numerous benefits. The increasing costs, increasing difficulty of extraction, and depletion of known fossil fuel reserves help to spur the development of such alternative fuel supplies. Efforts have been made to develop renewable energy fuels such as ethanol from corn grain or biodiesel from canola, rapeseed and other sources. The amount of biofuel that can be derived from food plant materials is often limited and the underlying increase in food commodity prices often negatively impacts food availability in developing countries and food prices in the developed world.

Efforts are underway to generate biofuels from non-food materials, such as cellulosic ethanol from wood pulp, corn stover or sugar cane bagasse. Algae and other photosynthetic microorganisms can provide feedstock for biofuel synthesis. Biofuel production from algae could permit productivities per unit of land area orders of magnitude higher than those of corn, rapeseed, canola, sugar cane, and other traditional crops.

Growing algae as a feedstock for biodiesel may involve growing the algae inside of closed bioreactors. Carbon, usually in the form of carbon dioxide (CO₂), is often added to the bioreactor media to support photosynthesis. Similarly, the process of photosynthesis liberates oxygen (O₂) which dissolves in the media. Relying on an open bioreactor exposed to ambient air in order to receive carbon dioxide from the air and vent the liberated oxygen to the air often does not yield enough carbon to support effective algae growth, due to the relatively low carbon dioxide content of air. Bubbling carbon dioxide directly into the bioreactor media may often involve a relatively low carbon dioxide absorption into the media, such that supplying the carbon dioxide often requires more energy than is produced by the algae growth. Using a complex membrane contactor to promote the absorption of carbon dioxide into the media often involves a relatively high expense, which also often requires a greater cost than the value of the energy produced through algae growth.

### SUMMARY

Embodiments of the present invention transfer CO₂ to the bioreactor media molecularly in a highly cost-effective manner. According to some embodiments of the present invention, porous and non-porous membranes are incorporated into a film-based photobioreactor to create a continuous or distributed contactor. Such membranes used to transfer the CO₂ into the media (e.g. water), or remove O₂ from the media, may be integrated directly into a plastic film reactor structure, according to embodiments of the present invention. This reduces cost, reduces a need for pumping, and reduces the size of the reactor (compared to a less efficient reactor) according to embodiments of the present invention. Such a configuration also permits a double use of the contactor material, which can function as part of the photobioreactor structure in addition to its function as a gas exchange membrane. According to some embodiments of the present invention, the membranes may comprise one or more chambers filled with a gas, one or more valves, a pressure source, and/or a means to control pressure within the chambers.

Any known species of algae or photosynthetic microorganisms may be grown in a photobioreactor and utilize such integrated membranes, according to embodiments of the present invention. According to some embodiments of the present invention species such as, but not limited to, Nannochloropsis oculata, Nannochloropsis sp., Nannochloropsis salina, Nannochloropsis gaditana, Tetraselmis suecica, Tetraselmis chuii, Chlorella sp., Chlorella salina, Chlorella protothecoides, Chlorella ellipsoidea, Chlorella emersonii, Chlorella minutissima, Chlorella pyrenoidosa, Chlorella sorokiniana, Chlorella vulgaris, Chroomonas slaina, Cyclotella cryptic, Cyclotella sp., Dunaliella tertiolecta, Dunaliella salina, Dunaliella bardawil, Botryococcus braunii, Euglena gracilis, Gymnodimium nelsoni, Haematococcus pluvialis, Isochrysis galbana, Monoraphidium minutium, Monoraphidium sp., Nannochloris, Neochloris oleoabundans, Nitzschia laevis, Onoraphidium sp., Pavlova lutheri, Phaeodactylum tricornutum, Porphyridium cruentum, Scenedesmus obliquus, Scenedesmus quadricaula, Scenedesmus sp., Stichococcus bacillaris, Stichococcus minor, Spirulina platensis, Thalassiosira sp., Chlamydomonas reinhardtii, Chlamydomonas sp., Chlamydomonas acidophila, Isochrysis sp., Phaeocystis, Aureococcus, Prochlorococcus, Synechococcus, Synechococcus elongatus, Synechococcus sp., Anacystis nidulans, Anacystis sp., Picochlorum oklahomensis, Picocystis sp. may be grown either separately or as a combination of species.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a photobioreactor system comprised of plastic film with two integrated membrane tubes and an integrated sparging tube, according to embodiments of the present invention.

FIG. 2 is a cross-sectional view of a photobioreactor system comprised of plastic film with two integrated membrane tubes and an integrated sparging tube, according to embodiments of the present invention.

FIG. 3 illustrates a photobioreactor system comprised of film with an integrated membrane tube and an integrated sparging tube, according to embodiments of the present invention.

FIG. 4 illustrates a cross-sectional view of a photobioreactor system comprised of film with an integrated membrane tube and an integrated sparging tube, according to embodiments of the present invention.

FIG. 5 illustrates a photobioreactor with an integrated membrane and an integrated sparging tube, according to embodiments of the present invention.

FIG. 6 illustrates a cross sectional view of a photobioreactor with multiple integrated membrane tubes and an integrated sparging tube, according to embodiments of the present invention.

FIG. 7 illustrates a cross sectional view of photobioreactor comprised of film with multiple integrated tubes, according to embodiments of the present invention.

FIG. 8 illustrates a photobioreactor system comprised of plastic film with an integrated membrane tube, an integrated sparging tube, and a bottom portion of the photobioreactor bag formed of a permeable membrane, according to embodiments of the present invention.

FIG. 9 illustrates a cross-sectional view of an alternative photobioreactor configuration comprised of plastic film with an integrated membrane tube, an integrated sparging tube and a portion of the photobioreactor bag formed of a permeable film, according to embodiments of the present invention.

FIG. 10 illustrates a method of construction for welding membrane tubes together, according to embodiments of the present invention.

FIG. 11 illustrates a final product of an integrated membrane tube made of two sheets of film welded in between two other layers, according to embodiments of the present invention.

FIG. 12 illustrates a method of construction in which two layers of membrane film trap another layer of thicker film to aid welding, according to embodiments of the present invention.

FIG. 13 illustrates membrane tubes in which two layers of membrane film trap another layer of thicker film, according to embodiments of the present invention.

FIG. 14 illustrates a cross sectional view of a product resulting from a construction in which two layers of membrane film trap another layer of thicker film to aid welding, according to embodiments of the present invention.

FIG. 15 illustrates a cross-sectional view of an alternate configuration with an integrated sparging tube in which permeable membrane tubes are integrated into a photobioreactor bag, according to embodiments of the present invention.

FIG. 16 illustrates a cross sectional view of an alternate configuration in which a photobioreactor bag is a membrane and gases transfer through the bag surface, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

Researchers are exploring growing algae as a feedstock for biodiesel. In many designs the algae is grown inside closed reactors comprised of glass or plastic. Examples of closed system bioreactors suitable for growth of algae and other microorganisms are described in U.S. patent application Ser. No. 11/871,728, filed Oct. 12, 2007, which is incorporated by reference herein in its entirety.

One approach for introducing CO₂ into the media, or the water in which the algae is grown, involves allowing the free surface of the media to be exposed to atmospheric air. Typical air contains approximately 0.038% CO₂ by volume. While such a configuration is relatively easy to implement, it does not allow for much carbon to be added to the media/water and therefore the effectiveness of the algae growth may not be as high in such circumstances.

Another approach to increase the carbon content of the water is to bubble, or sparge, gaseous CO₂ through the media. The CO₂ may be sparged through the media either in pure form or mixed with other gases, such as, for example, air. Bubbles formed will rise through the media and a portion of the CO₂ will be absorbed into the media, adding carbon and altering the pH content of the media. However, there is often not enough time for all of the CO₂ in the bubbles to be absorbed before these bubbles reach the top surface of the media. In many cases little CO₂ is absorbed and the non-absorbed CO₂ is expelled with the vent air, resulting in low uptake efficiency. The cost to preprocess and pump the CO₂ gas in such cases can be relatively high.

Other configurations increase the residence time of bubbles in the media before the bubbles reach the free surface. For example, CO₂ as bubbles may be injected at the bottom of a pipe oriented vertically with the media flowing from top to bottom, such that the average velocity of the media in the pipe is approximately the same, or slightly slower than, the velocity at which the bubbles rise. While this increases the residence time of the bubbles in the media, energy is expended in continuously pumping the fluid.

An alternative to bubbling CO₂ is to use porous or non-porous membranes or other materials that transfer the gas without bubbling, according to embodiments of the present invention. Two classes of materials can be used for this, for example. One class of such materials includes non-porous membranes that transfer CO₂ molecularly into the media rather than by bubbling it through the media. Various different non-porous membranes successfully distribute CO₂ into media for the purposes of growing algae. Such non-porous membranes may also be used in medical devices to oxygenate blood, or transfer other gases into liquids. Silicone rubber is one example of a non-porous membrane that has high permeability to CO₂ and other gases yet is effectively waterproof in the sense that water or media does not permeate through the membrane.

Another class of materials capable of gas exchange with a photobioreactor media are porous membranes that have very small holes, according to embodiments of the present invention. Such holes may be large enough to allow CO₂ molecules to penetrate through the membrane and form a gaseous CO₂ skin, or attached bubble, in direct contact with the media, while not large enough to permit bubbles to form, detach, and rise in the media, according to embodiments of the present invention. Such holes may also be sufficiently small so as to not allow liquids to pass through and may be essentially “waterproof” as well, according to embodiments of the present invention. Beneficial characteristics of a gas transfer membrane according to various embodiments of the present invention include a high surface area available to transfer CO₂ into the media, and a dimension to permit a sufficient time for the CO₂ gas to be absorbed.

Membrane materials can be built into assemblies that are often referred to as “membrane contactors,” in which large amounts of such materials are folded and mounted in a container to provide a high surface-area-to-contactor volume ratio. Such contactor shells may be made of hard plastic, metal or other rigid material. Such contactors permit liquid to be circulated over the membrane at high flow rates while gas flows on the other side of such membrane to further increase gas transfer. Contactors provide a relatively compact passage for gas transfer, although they can often be expensive. Contactors are often localized, and a pump or similar device is used to move the media to the device. This can be expensive from a capital and operating cost standpoint, and many algae are sensitive to the shear caused by pumping.

Some embodiments of the present invention involve photobioreactors used to grow algae for the production of biodiesel. In some embodiments, the bioreactors may be used to grow algae or other photosynthetic microorganisms and the membranes may be optimized to efficiently introduce carbon dioxide (CO₂), and/or remove dissolved oxygen (O₂) from the media in which the algae or other microorganisms are grown. Based on the disclosure provided herein, one skilled in the art will recognize that similar configurations can also be used to grow algae for other purposes, and/or to grow other microorganisms.

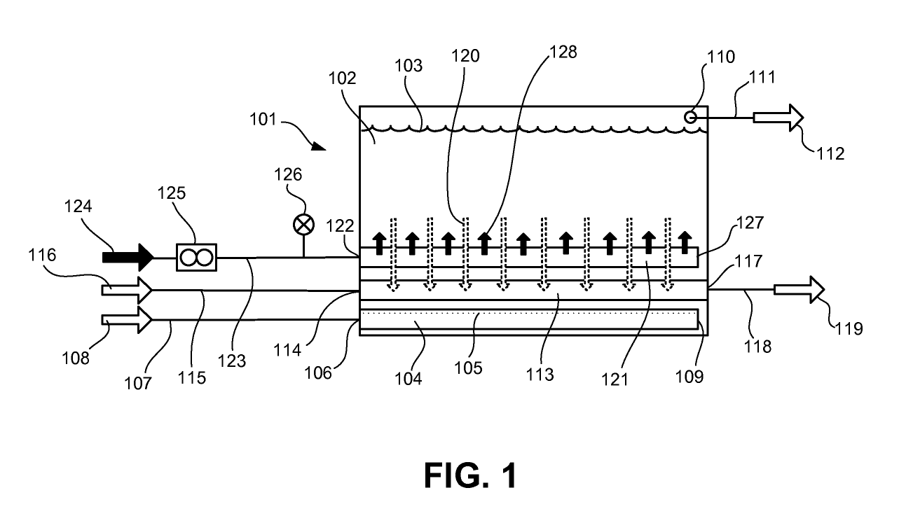

FIG. 1 illustrates an embodiment of an integrated membrane contactor. According to some embodiments of the present invention, a porous or non-porous membrane 121 may be integrated into a film based photobioreactor bag to add CO₂ to media 102. According to some embodiments of the present invention, multiple tubes may be heat welded from non-porous membranes and integrated into a basic bag structure. In FIG. 1, a tube 121 filled with CO₂ adds carbon to the media, a tube 113 filled with air removes dissolved oxygen (O₂) from the media, and another tube 104 filled with air sparges the system, according to embodiments of the present invention.

FIG. 1 depicts a photobioreactor 101 comprised of multiple sheets of plastic welded together. In this embodiment, the film is a symmetric composite film comprised of nylon “sandwiched” between two layers of low density polyethylene that were bonded to the nylon with tie layers. According to some embodiments of the present invention, the film is approximately 0.005 inches thick, eighteen inches tall and nine feet long. According to some embodiments of the present invention, the film (and thus the photobioreactor bag) is sixty inches long. According to other embodiments of the present invention, the film (and thus the photobioreactor bag) is two hundred fifty feet long. According to some embodiments of the present invention, the cross-sectional geometry of the photobioreactor bag is consistent and/or substantially similar along its length or most of its length. The film is thermally welded to form a hermetically sealed photobioreactor containing media 102; according to embodiments of the present invention, the top level surface 103 of the media 102 is below the top of the photobioreactor bag 101 such that the media 102 has a free surface 103 in it to allow air to collect. According to some embodiments of the present invention, the bag 101 is welded using a thermal impulse welder; according to other embodiments other methods of welding may be used with similar results, such as, for example, constant temperature thermal welding, RF welding, ultrasonic welding or other means. According to some embodiments of the present invention, the components of the photobioreactor bag 101 may be attached with adhesive, may be melted along the weld lines, may be stitched and/or stapled, and/or may be crimped together in a way which minimizes escape of the liquid media 102 or other system fluids.

The photobioreactor includes an integrated air tube 104 that is thermally welded into the film of the bioreactor 101, according to embodiments of the present invention. The integrated air tube 104 may be constructed with a 0.0035″ thick composite plastic also comprised of low density polyethylene, nylon and tie layers used to bond the nylon to the polyethylene, according to embodiments of the present invention. The integrated air tube 104 may be used for sparging the media 102; such sparging may be accomplished as the gas, usually air, leaves the air tube 104 through sparging holes 105. These holes are approximately 0.010 inches in diameter and are spaced approximately 0.5 inches apart, according to embodiments of the present invention. The holes 105 may be cut using a laser, or alternatively using mechanical punches or other hole creation methods.

The air tube 104 is fed from a fitting 106 that is connected to the air feed line 107, which in turn is connected to a source 108 of higher pressure air or gas mixture. Typical sparge pressures are two to three pounds per square inch gage (“psig”). The other (far) end of the air tube is sealed with another thermal weld 109, according to embodiments of the present invention. The sparge air rises as bubbles through the media 102 and leaves the photobioreactor 101 through the exhaust port 110, according to embodiments of the present invention. The exhaust port 110 may be thermally welded into the film of the bioreactor 101, according to embodiments of the present invention. The exhaust port 110 is in fluid communication with an exhaust line 111, and the exhaust line 111 is in fluid communication with a device 112 which regulates the backpressure in the photobioreactor bag 101.

The photobioreactor may also include an integrated membrane tube 113 used to allow dissolved oxygen in the media 102 to permeate through the media 102 and into the gas inside the tube 113, according to embodiments of the present invention. The tube 113 may be composed of 0.0015 inch thick composite film; in some cases the tube 113 may be thermally welded to the outer bag of the photobioreactor 101 such that the film of the tube 113 and the photobioreactor bag 101 form an integrated unit with the tube 113 being approximately one inch in diameter, according to embodiments of the present invention. The inside of the membrane tube 113 does not communicate with the media 102 inside the photobioreactor 101, other than to permit gas transfer, according to embodiments of the present invention. In one embodiment the tube 113 is made from a non-porous permeable membrane comprised of polyethylene and/or other plastics. Both non-porous and porous membranes may be used in the development of the photobioreactor 101 system, according to embodiments of the present invention. While the rates at which the different materials transfer the oxygen vary, satisfactory results are obtained with a variety of materials including numerous composite films both porous and nonporous, spun polyethylene (Tyvek), and/or silicone rubber, according to embodiments of the present invention. According to some embodiments of the present invention, non-porous membranes may be formed with a Sealed Air HP2700 (or 10K) film. According to some embodiments of the present invention, a porous membrane may be formed with an Aptra PP Microporous UV8 film manufactured by RKW US, a TYVEK 4058B and/or TYVEK 1025D film material manufactured by Dupont, a microporous film/non-woven laminate manufactured by Tredegar, a 4560-0400E-C microporous film and/or 2500-0400E-C microporous film manufactured by Celgard, and/or a UPHP000HC 0.45 UM UPE Membrane manufactured by Entegris.

As used herein, the phrase “membrane tube” is used in its broadest sense to refer to an enclosure and/or partial enclosure and/or liquid/gas interface comprised of a porous or nonporous membrane material which permits the transfer of one or more gases across the membrane, from an area containing liquid to an area containing gas or vice versa, according to embodiments of the present invention. A membrane tube need not be tubular, and need not include a cross section of uniform shape and/or diameter and/or dimension. A membrane tube need not have more than one opening. For example, a membrane tube may be a tube, a pocket, a line, a bag, a sleeve, and/or an enclosure formed at least partially of a gas permeable membrane, according to embodiments of the present invention.

The oxygen removal tube 113 is fed from a port 114 which is connected to (e.g. in fluid communication with) a feed line 115 which is fed by a supply of gas 116 used to strip the oxygen out of the media 102, according to embodiments of the present invention. The gas supply 116 may consist of air, air enriched with nitrogen, pure nitrogen, and/or other gases capable of drawing oxygen out of the media 102 and through tube 113. The stripping gas leaves the photobioreactor 101 through an exhaust port 117 in fluid communication with the membrane tube 113, through an exhaust tube 118, and into a backpressure control device 119, according to embodiments of the present invention. The dissolved oxygen leaving the media 102 and flowing into the membrane tube is depicted by arrows 120, according to embodiments of the present invention.

The photobioreactor 101 also includes a second membrane tube 121 constructed with a flexible film in a manner similar to tube 113, according to embodiments of the present invention. According to embodiments of the present invention, the fluid inside tube 121 does not communicate with the media 102 other than to permit gas transfer; in other words, the membrane tube 121 does not permit entry of the media 102 into the tube 121. Membrane tube 121 permits transfer of CO₂ from inside the membrane tube 121 to the media 102, according to embodiments of the present invention. The CO₂ membrane tube 121 is fed through a port 122 and a CO₂ feed line 123, according to embodiments of the present invention. The flow to the line 123 and therefore the membrane tube 121 is controlled with a flow control valve 125, which in turn is fed by a source 124 of CO₂, according to embodiments of the present invention. A pressure sensor 126 may be used to measure the pressure in the membrane tube 121. The other (far) end of the membrane tube 121 is sealed with a thermal weld 127, according to embodiments of the present invention. Arrows 128 illustrate the transfer of carbon dioxide from tube 121 into media 102, according to embodiments of the present invention.

The amount of CO₂ transferred to the media 102 from inside the tube 121 is a function of the material properties, the surface area of the membrane tube 121 and the difference in the partial pressures in the gas inside the membrane tube 121 and the equivalent partial pressure in the media 102. Consequently, the amount of CO₂ added to the media 102 can be controlled by adjusting the pressure inside the membrane tube 121, according to embodiments of the present invention. According to some embodiments of the present invention, pressures within tube 121 ranged from approximately one to ten psig.

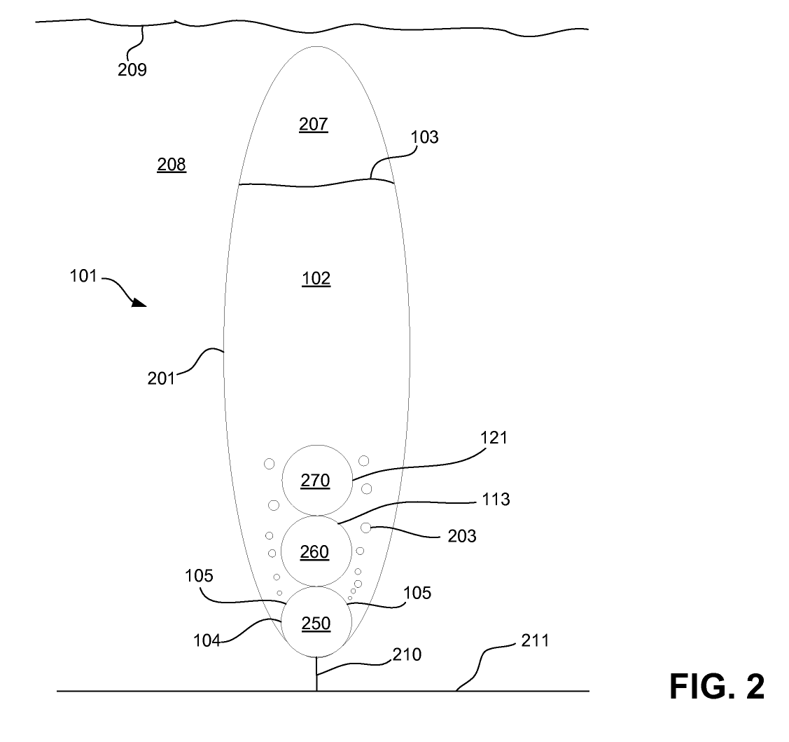

FIG. 2 shows a side cross-sectional view of the embodiment depicted in FIG. 1. The photobioreactor 101 includes an outer layer of film 201, and an integrated tube 104 also comprising film which is used to sparge the media 102 in the form of bubbles 203. FIG. 2 also shows an integrated membrane tube 113 used to remove dissolved oxygen from the media 102 and a second integrated membrane tube 121 used to supply CO₂ to the media 102 molecularly. The top level 103 of the media 102 inside the bag 101 is such that the bag 101 includes an area 207 above the media 102 in which air and other gases can collect. The entire photobioreactor 101 may be immersed in a water bath 208 with the top water level 209 of the water bath 208 higher than the top of the photobioreactor 101 according to embodiments of the present invention. The photobioreactor may be restricted by tether 210 fastened to the ground or other underlying surface 211 to prevent it from turning over or floating to the surface due to the buoyancy of the trapped air. According to other embodiments of the present invention, some or all of the photobioreactor extends above the free surface 209 of the water bath 208.

As discussed above, tube 104 may include air or another sparge gas 250; membrane tube 113 may include a gas with a relatively low oxygen content 260 (e.g. air, air enriched with nitrogen, pure nitrogen, and/or other gases in which the partial pressure of oxygen is lower than the equivalent partial pressure of oxygen in the media, causing the oxygen to diffuse from the media 102 and through tube 113); and membrane tube 121 may include carbon dioxide or a carbon enriched gas 270, according to embodiments of the present invention.

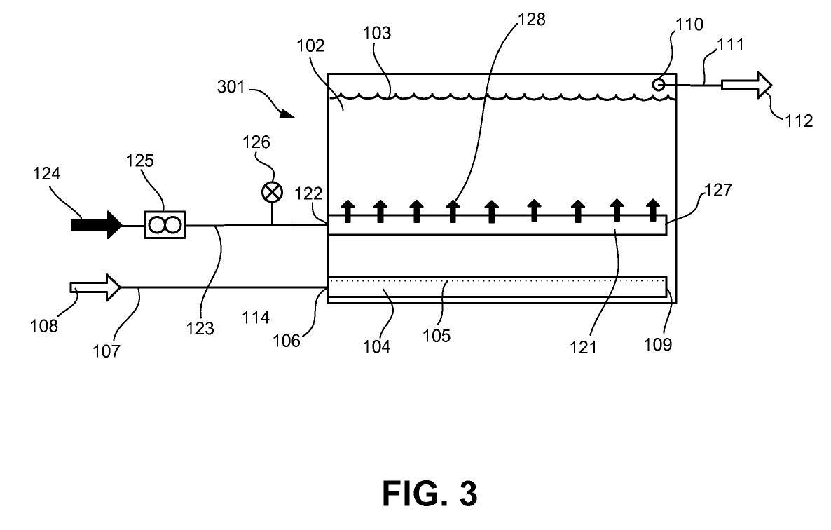

FIG. 3 illustrates an alternative photobioreactor 301 embodiment of the present invention, which is similar to the embodiment of FIGS. 1 and 2 except that it lacks a membrane tube 113 for removing dissolved oxygen from the media 102. According to some embodiments of the present invention, dissolved oxygen from the media 102 (which is a byproduct of photosynthesis of algae growth, for example) leaves the media 102 and exits through vent 110. Removing excess oxygen contributes to efficient culture growth and prevents buildup of a toxic level of oxygen, according to embodiments of the present invention. According to some embodiments of the present invention, the photobioreactor 301 bag 201 itself is capable of permitting the dissolved oxygen to pass through the photobioreactor 301 bag 201 and into a fluid surrounding or partially surrounding the photobioreactor 301 bag 201.

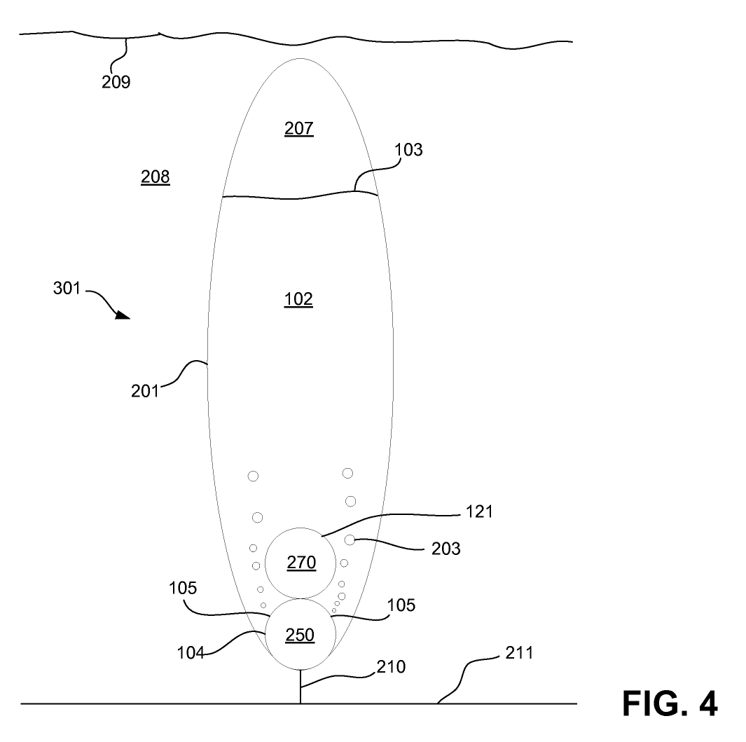

FIG. 4 illustrates a side cross-sectional view of the photobioreactor 301 of FIG. 3, according to embodiments of the present invention.

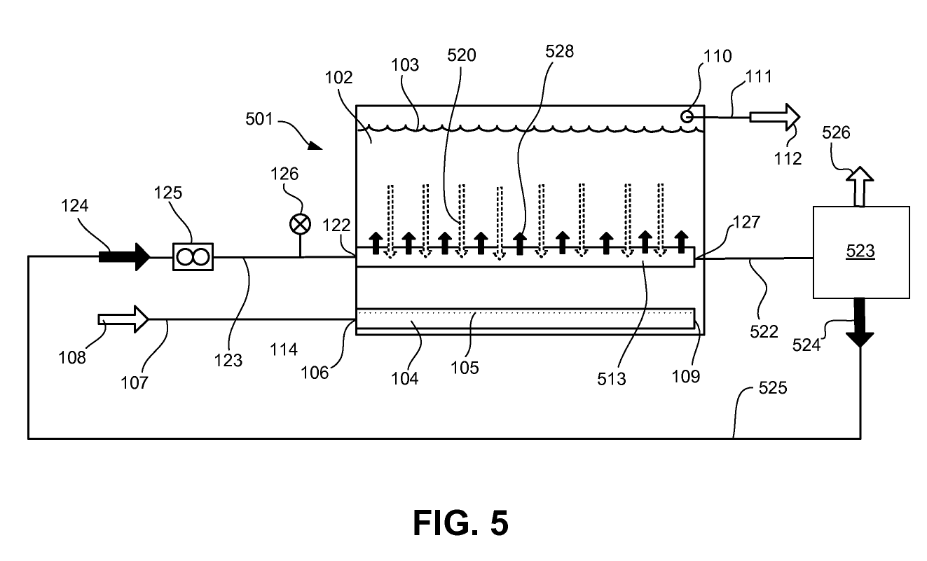

FIG. 5 shows an alternate photobioreactor 501 in which an integrated membrane tube 513 is used both to distribute CO₂ to the media 102 and to remove O₂ from the media 102, according to embodiments of the present invention. According to some embodiments of the present invention, the different partial pressures of oxygen within the tube 513 and in the media 102, and of carbon dioxide within the tube 513 and in the media 102, enable transfer of different gases in different directions as shown in FIG. 5.

According to some embodiments of the present invention, the photobioreactor also includes a membrane tube 513 comprised of flexible film comprised of a gas permeable membrane. The membrane is a non-porous plastic composite film, according to embodiments of the present invention. This integrated membrane tube 513 is constructed so that the fluid (e.g. gas) within the tube 513 does not communicate with the media 102. In other words, gas exchange occurs across the tube 513 but the media is not able to enter the tube 513, according to embodiments of the present invention. The membrane tube 513 is used to transfer CO₂ from inside the membrane tube 513 to the media 502 and to remove dissolved oxygen from the media 502, according to embodiments of the present invention. Carbon dioxide is fed to the membrane tube 513 through a port 122 and a CO₂ feed line 123. The flow to the line 123 and therefore the membrane tube 513 is controlled with a flow control valve 125 which is fed by a source 124 of CO₂. A pressure sensor 126 is used to measure the pressure in the membrane tube, according to embodiments of the present invention. The far end of the membrane tube 513 is not closed but has a fitting 127 such that the gas inside the tube 513 can flow out of the membrane tube 513, according to embodiments of the present invention. The flow of CO₂ into the media 102 is depicted with arrow 528 and the flow of O₂ from the media is depicted with arrow 520, according to embodiments of the present invention.

When the mixture gas inside the membrane tube 513 leaves the photobioreactor 501 through fitting 127, it travels through a line 522 and into an oxygen/carbon dioxide separator 523 which separates the O₂ gas from the CO₂ gas, according to embodiments of the present invention. The O₂ removed from the mixture gas is exhausted from the separator 523 as indicated by arrow 526 and the CO₂ from the mixture gas is returned to the CO₂ source 124 via CO₂ recirculation line 525, as indicated by arrow 524, according to embodiments of the present invention. In this way, a single membrane tube 513, or multiple tubes with the same or a similar gas in them, simultaneously adds CO₂ to the media 102 and removes O₂ from the media 102, according to embodiments of the present invention. The oxygen removed as indicated by arrow 526 may be stored and/or used in other applications, as a byproduct of the photosynthesis process, according to embodiments of the present invention.

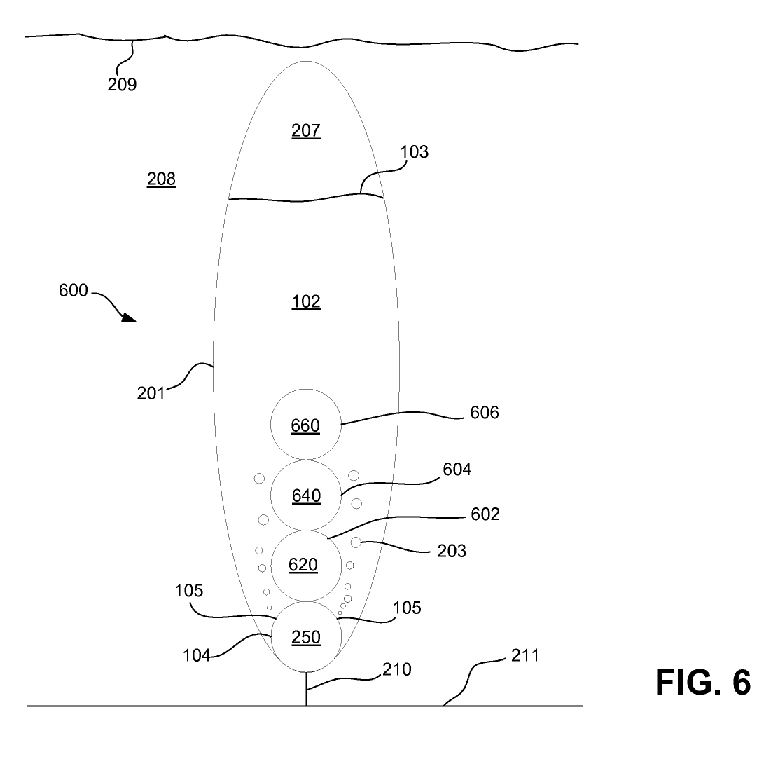

FIG. 6 illustrates a cross-sectional view of a photobioreactor 600 including multiple membrane tubes, according to embodiments of the present invention. Including multiple membrane tubes with photobioreactor provides a larger surface area to increase mass flow of gas transfer without increasing the membrane tube diameter, according to embodiments of the present invention. Here a film bag 201 includes an integrated film sparging tube 104 which creates bubbles 203 in the media 102. Three membrane tubes 602, 604 and 606 are integrated into the bag 201 by welding the film together to form membrane tubes 602, 604, 606 according to embodiments of the present invention. The membrane tubes 602, 604, 606 can be used in various combinations and numbers to increase the surface area of the membranes used for transferring O₂ and/or CO₂.

According to some embodiments of the present invention, two of the tubes 602, 604, 606 are used to remove O₂ from the media 102 and one of the tubes 602, 604, 606 is used to add the CO₂ to the media 102. According to some embodiments of the present invention, the membrane tubes 602, 604, 606 are constructed of similar material; according to other embodiments of the present invention, membrane tubes 602, 604, 606 may be constructed of different materials or a combination of materials. The fluids 620, 640, and 660 may be selected to be the same, or different, in order to remove oxygen from and/or add carbon dioxide to the media 102, as described above, according to embodiments of the present invention. For example, fluids 620 and 640 may be air or nitrogen enriched air to remove oxygen from the media 102, and fluid 660 may be carbon dioxide in order to add carbon dioxide to the media 102, according to embodiments of the present invention. Based on the disclosure herein, one of ordinary skill in the art will appreciate the number of different fluids 620, 640, 660 and the number of different permeable membrane materials and configurations that may be used for tubes 602, 604, 606 to achieve similar results, according to embodiments of the present invention.

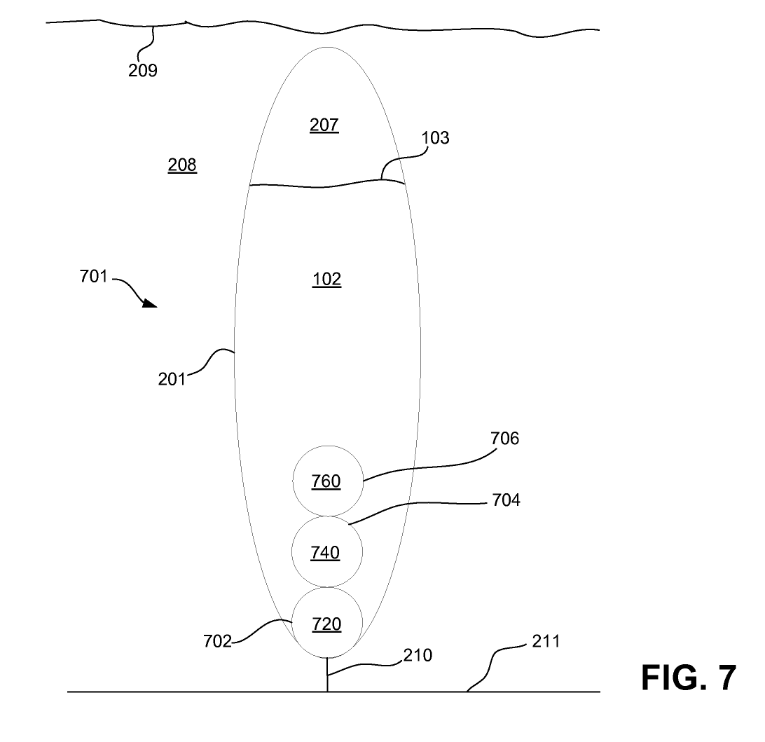

FIG. 7 illustrates an alternative photobioreactor 701 including integrated membrane tubes but not a sparging tube, according to embodiments of the present invention. Photobioreactor 701 may be composed of plastic film 201 and include integrated membrane tubes 702, 704 and 706 used to distribute CO₂ into the media 102, according to embodiments of the present invention. Membrane tubes 702, 704 and 706 may be constructed with thermally welded composite film, according to embodiments of the present invention. According to some embodiments of the present invention, membrane tubes 702, 704 and 706 may be connected together to permit fluid communication between two or more of the tubes 702, 704 and 706. Such fluid communication may be achieved either via the welding process or by adding lines outside the bag 201, for example.

According to some embodiments of the present invention, two of the tubes 702, 704, 706 are used to remove O₂ from the media 102 and one of the tubes 702, 704, 706 is used to add the CO₂ to the media 102. According to some embodiments of the present invention, the membrane tubes 702, 704, 706 are constructed of similar material; according to other embodiments of the present invention, membrane tubes 702, 704, 706 may be constructed of different materials or a combination of materials. The fluids 720, 740, and 760 may be selected to be the same, or different, in order to remove oxygen from and/or add carbon dioxide to the media 102, as described above, according to embodiments of the present invention. For example, fluids 720 and 740 may be air or nitrogen enriched air to remove oxygen from the media 102, and fluid 760 may be carbon dioxide in order to add carbon dioxide to the media 102, according to embodiments of the present invention. Based on the disclosure herein, one of ordinary skill in the art will appreciate the number of different fluids 720, 740, 760 and the number of different permeable membrane materials and configurations that may be used for tubes 702, 704, 706 to achieve similar results, according to embodiments of the present invention.

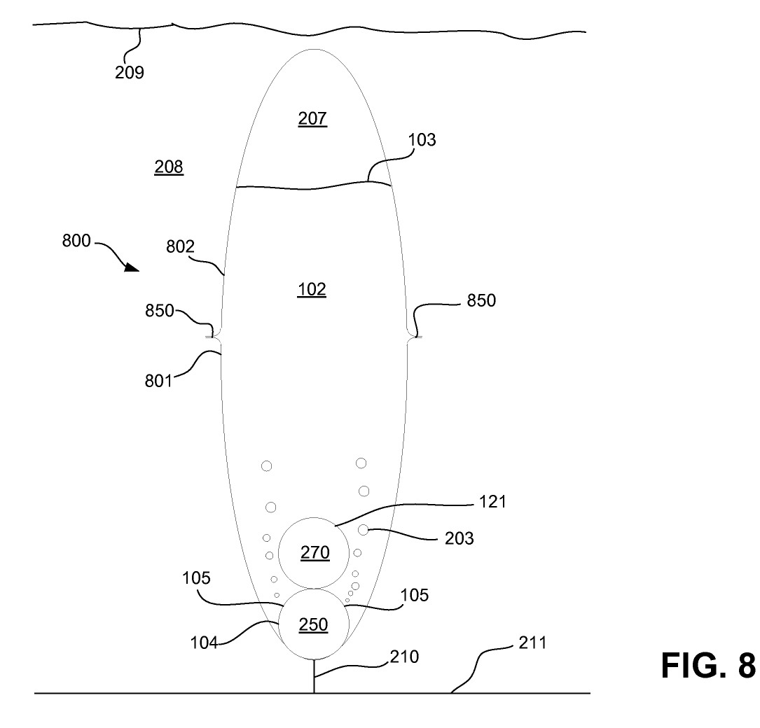

According to some embodiments of the present invention, all or a portion of the photobioreactor bag itself may include a higher permeability membrane in order to increase the amount of surface area available for gas transfer. Some higher permeability membranes do not pass light very well; this could affect the reactor performance in some cases, if the high permeability membrane is used for the exterior of the reactor. According to some embodiments, the reactor is configured such that the effect of the reduced light transmission is minimized. FIG. 8 illustrates a photobioreactor 800 comprised of a film 802 that is transparent and allows most of the sunlight to pass through, and a permeable membrane 801 that may not be very transparent, according to embodiments of the present invention. The film 802 and the membrane 801 may be welded together to form an integrated membrane/photobioreactor. Exemplary weld/seam locations are shown at 850. The embodiment shown in FIG. 8 includes an integrated sparging tube 104 also made from flexible film welded to the reactor film walls to distribute to gas in the form of bubbles 203, according to embodiments of the present invention. Photobioreactor 800 also includes an integrated membrane tube 121 used to transfer CO₂ or other gases to the media 102. Membrane 801 permits the diffused oxygen within the media 102 to transfer from the media 102, across the membrane 801, and out into the surrounding fluid 208, according to embodiments of the present invention. Photobioreactor 800 is shown supported in a bath of water 208, with water level 209, but according to other embodiments of the present invention, photobioreactor 800 need not be supported in water, and according to yet other embodiments of the present invention, the water level 209 may be below the top of the reactor 800. Reactor 800 is tethered to the bottom of the basin 211 using a tether 210, although various alternative methods could be employed to tether the bag 800 including, but not limited to, plastic film, film with bars, wire ties, cords, flexible bars, ballast and/or weights.

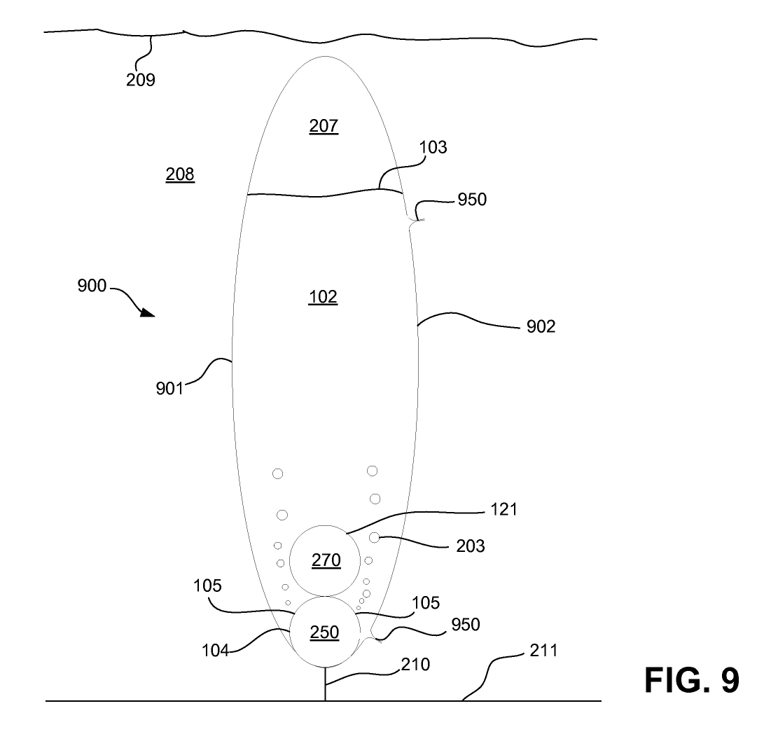

FIG. 9 illustrates a cross-sectional view of a photobioreactor 900 with a highly permeable membrane 902 located on one side of the bag 900. The permeable membrane 902 permits dissolved oxygen to pass from the media 102 and into the fluid surrounding the bag 900. Based on the disclosure provided herein, one of ordinary skill in the art will appreciate that numerous alternative sizes and placements of the permeable membrane 902 may be made within the photobioreactor 900, according to embodiments of the present invention. For example, the membrane 902 may be placed on the back side of the reactor 900 that is not facing the sun so minimal light is blocked. According to some embodiments of the present invention, the membrane 902 is placed on the side of the reactor 900 facing the sun in order to block the direct sunlight. The location and size of the permeable membrane 902 can be varied to match the application. The size of the membrane may also be varied to cover anywhere from a very small percentage of the reactor 900 surface area (for example 10%), to as much as 100% of the reactor 900 outer surface area. According to some embodiments of the present invention, multiple different sections and/or “patches” of membrane 902 may be used in the photobioreactor 900. The reactor 900 is made of flexible film 901 that is highly transparent and a membrane 902 that is highly permeable, but perhaps not as transparent of that of the base reactor film 901, according to embodiments of the present invention. The photobioreactor is shown with a sparging tube 104, although the sparging tube 104 is absent from reactor 900 in other embodiments.

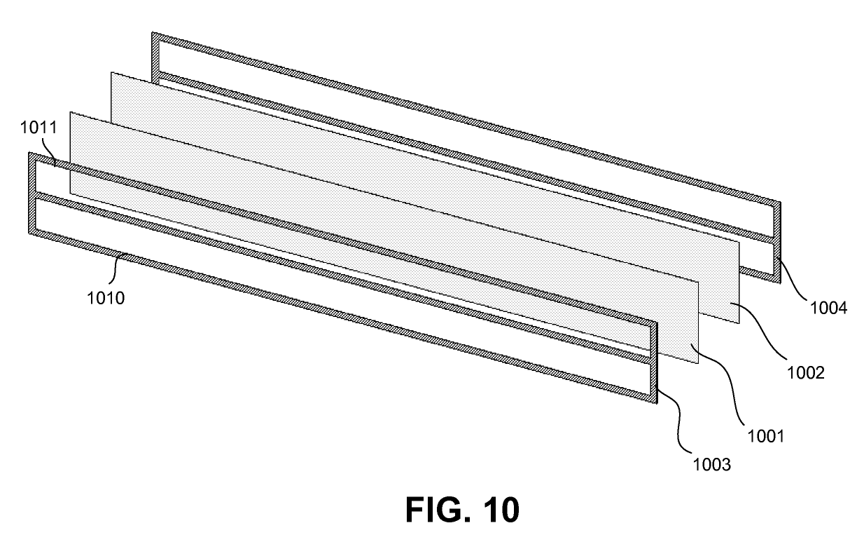

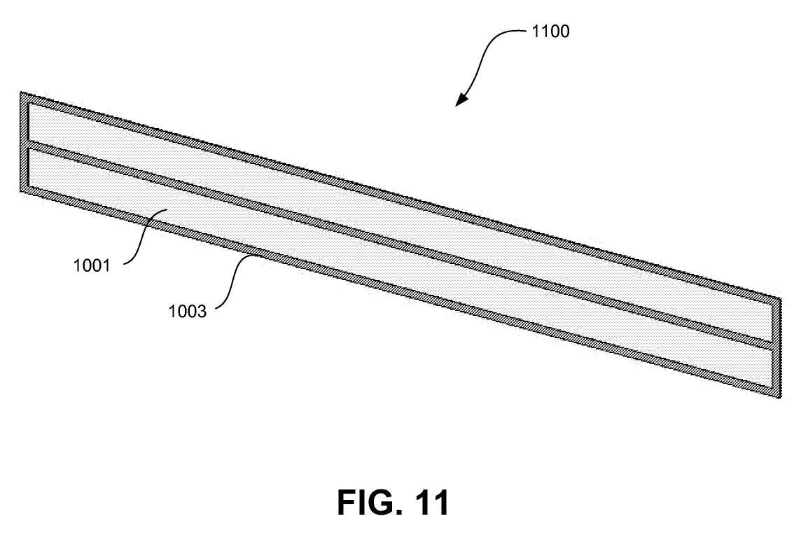

Numerous methods may be used to manufacture membranes integrated into film bioreactors. One method of doing this is to weld the different layers of film together to form a film photobioreactor, where portions are made of regular film and other sections are made of porous membranes to permit gas transfer, according to embodiments of the present invention. FIG. 9 illustrates a weld 950 location, according to embodiments of the present invention. Many different methods of welding the various layers and/or membranes together can be employed including, but not limited to, constant temperature thermal welding, impulse welding, Radio Frequency (RF) welding, and ultrasonic welding. Often, the membranes are very thin, or are made of materials that may be difficult to weld. Other means to join various films may be employed, such as, for example, using adhesives, glues, hot melt glues and/or by mechanically pressing the film to other materials with sufficient contact pressure to create a hermetic seal therebetween, according to embodiments of the present invention. FIGS. 10 and 11 illustrate a method for integrating permeable membranes into a film based photobioreactor by thermally welding, a process which also may be used for the other welding and joining methods discussed above, according to embodiments of the present invention.

FIG. 10 shows one layer 1001 of a non-porous permeable membrane. The membrane 1001 may be approximately 0.0015 inches thick, twenty-two inches long, and four inches tall, according to embodiments of the present invention. A second layer 1002 of similar membrane material is placed on top of the first film 1001, according to embodiments of the present invention. These two layers 1001, 1002 may be welded together to form two tubes that can contain a desired gas. Two tubes can be used rather than one if the desired gas transfer surface area is larger than could be achieved using one larger tube, and the tube stresses increase as the diameter increases, according to embodiments of the present invention. If the diameter is too high the welds may be more susceptible to failure, or the material itself may be more susceptible to failure. As shown in FIG. 10, the surface area of the layers 1003, 1004 is much less than the surface area of layers 1001, 1002, because the surface area of layers 1003, 1004 is configured to correspond generally to the weld locations between the layers and to provide a “buffer zone” around the welds to prevent and/or minimize damage to the more heat and/or stress sensitive membrane layers 1001, 1002, according to embodiments of the present invention. As such, layer 1003 includes membrane windows 1010, 1011 cut out or formed into the layer, which correspond generally to the locations at which the underlying membrane layer 1001 will be exposed in the final membrane tube, according to embodiments of the present invention.

CO₂ with pressures ranging from zero to as much as ten pounds per square inch or more may be used, according to embodiments of the present invention. In order to facilitate the welding, two outside pieces 1003, 1004 of thicker and more weldable film may be cut so that there is plastic at the desired locations of the welds. The outside pieces 1003, 1004 are configured to reinforce the weld locations of the membrane layers 1001, 1002 without adding too much plastic in the areas which do not need as much reinforcement. Adding too much heavier outer plastic can make the photobioreactors heavier for transport and may adversely affect the operation of the photobioreactor by reducing the amount of light that gets inside the reactor. Outside layers of film 1003, 1004 used to facilitate the welding may be made from 0.005 inch thick composite film that includes layers of polyethylene, nylon and “tie” layers between the polyethylene and nylon layers. The four layers 1001-1004 may be placed on top of each other as shown and welded using an impulse welder; during welding, the plastic from the outside welding layers 1003, 1004 melts and flows against the membrane layers 1001, 1002, according to embodiments of the present invention. This helps make a solid joint and reduces the possibility for holes or flaws in the weld joint, according to embodiments of the present invention. A similar method may also be used to join porous membranes consisting of spun polyethylene, and can be very effective with such materials as they tend to shrink and pull away from the weld area when exposed to heat. The outer layers 1003, 1004 also reduce the temperature experienced by the membranes 1001, 1002, further helping to control the welding process.

FIG. 11 shows final product 1100 made with the welding process described with respect to FIG. 10. Here two layers of membrane film 1001 have been welded between two layers of reinforcement plastic 1003 to form a sealed tube such as, for example, a double membrane tube configuration for placing within an outer bag 201 to transfer carbon dioxide to media 102, according to embodiments of the present invention. Based on the disclosure provided herein, one of ordinary skill in the art will appreciate that the outside reinforcement layers 1003, 1004 could be part of the photobioreactor structure so that little if any additional film is required to achieve an integrated photobioreactor integrated with permeable membranes, according to embodiments of the present invention.

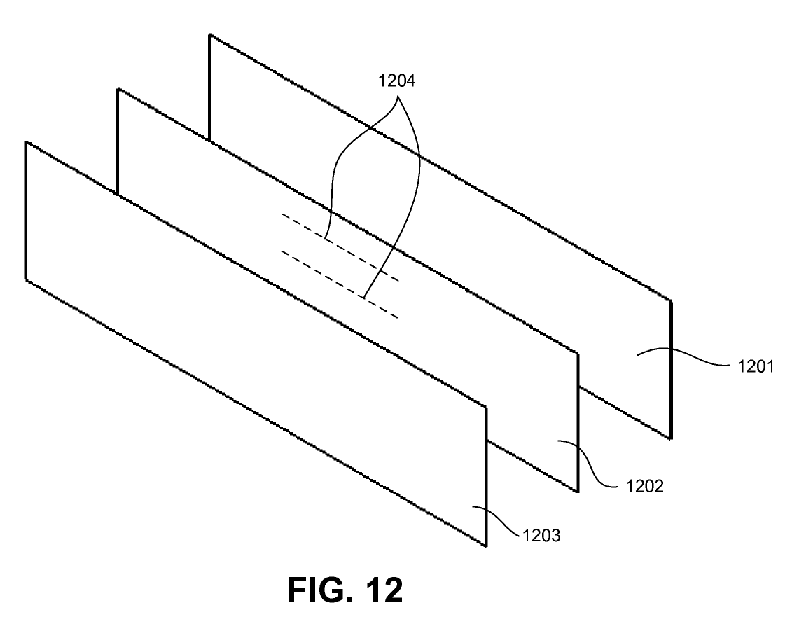

FIG. 12 shows an alternate method for constructing a film photobioreactor that has integrated permeable membranes welded into it, according to embodiments of the present invention. An inner welding reinforcement layer 1202 of film is sandwiched between two outer layers 1201, 1203 of permeable membrane, according to embodiments of the present invention. The outer film may be a non-porous composite film with a thickness of approximately 0.0015 inches. The inner welding reinforcement layer 1202 may be made of 0.0055 inch thick composite film, according to embodiments of the present invention. All three layers 1201, 1202, 1203 are approximately twenty-two inches long and approximately six inches tall, according to an embodiment of the present invention. The welding reinforcement layer 1202 has openings in the form of slits 1204 formed in it to allow the volume created between layers 1201 and 1202 to communicate with the volume created between layers 1202 and 1203, according to embodiments of the present invention. These openings 1204 can be of various shape and/or length and of sufficient area to permit passage of the gas between the two volumes, according to embodiments of the present invention.

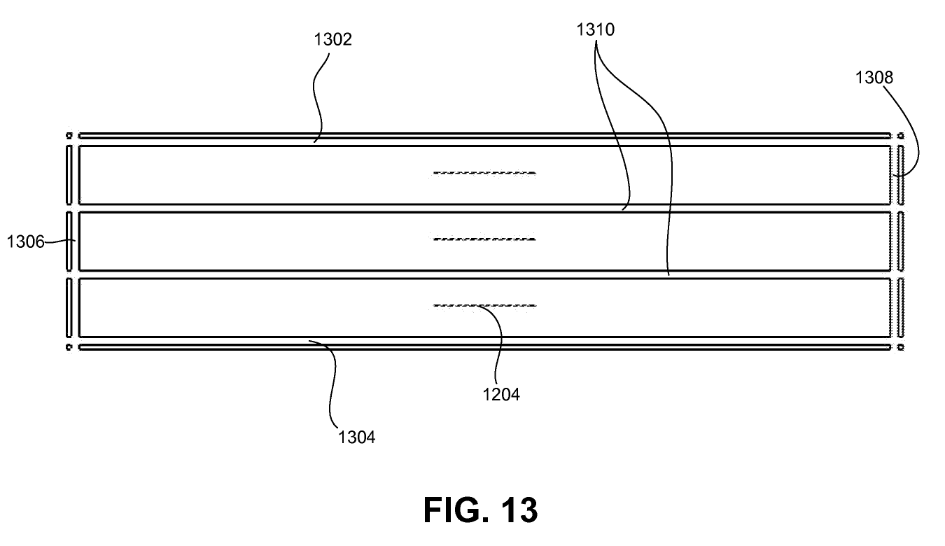

FIG. 13 shows a plan view of the three layers 1201, 1202, 1203 of film and corresponding weld locations, according to embodiments of the present invention. A top weld 1302, end welds 1306 and 1308 and a bottom weld 1304 are shown, according to embodiments of the present invention. Intermediate weld locations 1310 are also shown, according to embodiments of the present invention. FIG. 13 also shows a location of the slits 1204 in the middle welding reinforcement layer.

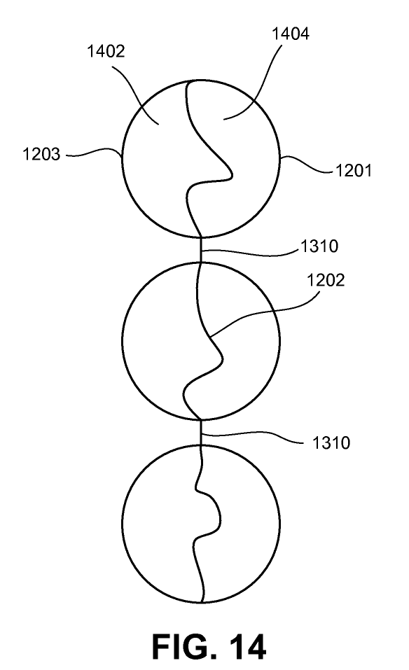

FIG. 14 illustrates a cross-sectional view of a sealed membrane tube using a center welding reinforcement layer 1202, according to embodiments of the present invention. Here two layers of permeable membrane 1201 and 1203 sandwich a welding reinforcement layer 1202 and are welded together, according to embodiments of the present invention. The middle welding reinforcement layer 1202 has slits in it that allow the two sides 1402, 1404 of that layer to act as one volume, according to embodiments of the present invention. Such a method of construction is quick to manufacture, the extra layer 1202 is not in an area that will block much light, and it provides a robust, easy-to-weld configuration, according to embodiments of the present invention.

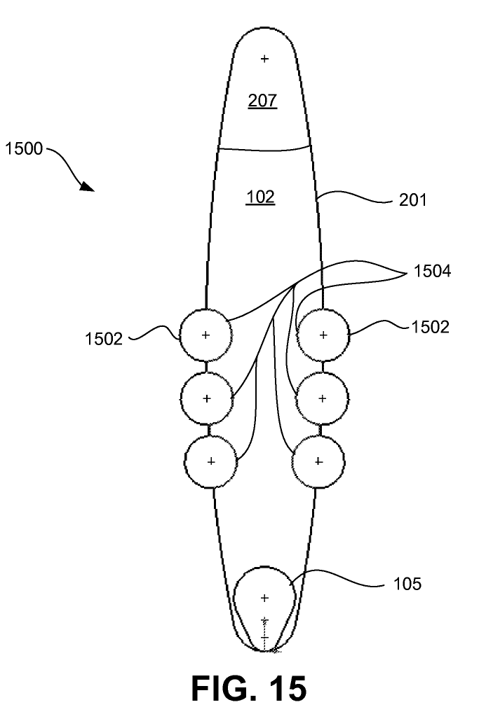

FIG. 15 illustrates an alternate embodiment of a photobioreactor 1500 with permeable membranes 1504 integrated into a film based photobioreactor 1500, according to embodiments of the present invention. In this embodiment the outer layer 201 of the photobioreactor 1500 is comprised of composite film approximately 0.005 inches thick, according to embodiments of the present invention. Welded to the inside of the photobioreactor film are separate layers 1504 of permeable membrane, such that sealed membrane tubes 1502 are made with the permeable membrane inside the photobioreactor, according to embodiments of the present invention. According to some embodiments of the present invention, the permeable membrane 1504 is approximately 0.0015 inches thick, and each layer is approximately twenty-two inches long and the entire photobioreactor 1500 is approximately eighteen inches tall. The volume inside the tubes 1502 can be filled with a gas at pressure above the static fluid pressure in the reactor 1500 such that the gas will be transferred into the fluid (media 102), according to embodiments of the present invention. An air tube 105 may be included for sparging to the media 102, according to embodiments of the present invention.

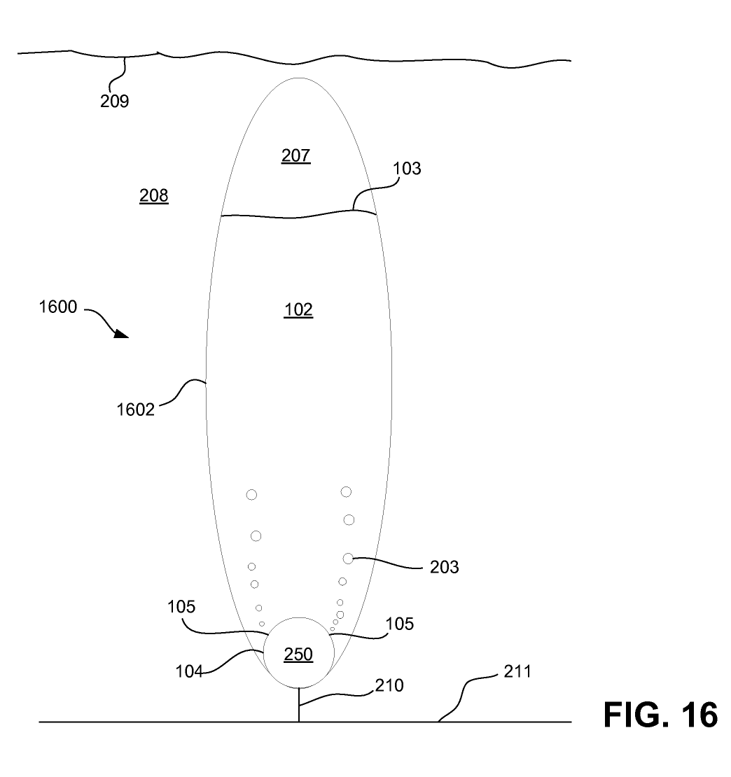

FIG. 16 also illustrates a photobioreactor 1600 in which the entire outer film 1602 is constructed of a permeable membrane to allow gases dissolved in the media 102 inside the reactor 1600 to be transferred through the membrane film 1602 and into the bath water 208 outside the reactor 1600, according to embodiments of the present invention. Such a configuration provides for a large amount of surface area without using additional film material, according to embodiments of the present invention. The permeable membrane 1602 transfers dissolved oxygen (O₂) from the inside media 102 where it could be detrimental to the growth of the organisms to the water 208 outside, according to embodiments of the present invention. Similarly, gases that are desired in the media 102 inside the reactor 1600 could be transferred from the bath water 208 through the membrane film 1602 and into the media 102, according to embodiments of the present invention. Carbon dioxide (CO₂) is one such example of a gas that might be desired in the media 102. The concentration of a gas dissolved in the bath water 208 may be increased and/or controlled to increase the rate of transfer of gas through the bag 1602 and into the media 102, according to embodiments of the present invention.

FIG. 16 illustrates a photobioreactor 1600 comprised of permeable film 1602, sparging tube 104 that may, or may not, be made of permeable film, to provide gas in the form of bubbles 203 to the media 102 which is at level 103. A volume 207 above the media 102 is provided to allow gases to collect and move down the length of the reactor 1600 where they can leave, according to embodiments of the present invention.

According to some embodiments of the present invention, the membrane tube includes a single opening in fluid communication with a gas source; for example, the membrane tube 121 of FIG. 3 includes a single port 122 in fluid communication with CO₂ source 124, according to embodiments of the present invention. According to other embodiments of the present invention, the membrane tube includes a first port in fluid communication with a gas source and an exhaust port through which the gas flows after flowing through the reactor bag; for example, the membrane tube 513 of FIG. 5 includes a first port 122 in fluid communication with CO₂ source 124 and an exhaust port 127 through which the CO₂ flows after flowing through the photobioreactor 501, according to embodiments of the present invention.

According to some embodiments of the present invention, a photobioreactor operates in an open loop condition to provide stable pH control with accuracy, by employing a method to estimate the growth rate from the required pressure in the membrane bag if it were a stable membrane bag. According to such embodiments, a photobioreactor includes a membrane tube integrated into the reactor to introduce CO₂ to the system. According to some embodiments of the present invention, the diffusion rate for CO₂ into the media may be primarily driven by the difference in partial pressure (or equivalent partial pressure if in liquids) between the media and the gas inside the tube. At certain pH values, as CO₂ is dissolved into the media the pH will become lower, all other things held constant, and it will rise as the CO₂ is depleted from the media; this results in the diffusion of the CO₂ through the membrane increasing as the pH rises, and decreasing as it lowers. According to embodiments of the present invention, a stable membrane may be included in the photobioreactor that will tend to automatically converge to a certain pH that would be a function of media content, cell growth rate, physical materials and configuration of the membrane (exposed surface area and permeability if the membrane material) and the pressure in the tube, according to embodiments of the present invention.

A membrane area can be selected such that the diffusion rate for a given partial pressure differential of CO₂ across the membrane will provide the desired amount of carbon to the media to maintain the pH. According to some embodiments of the present invention, if the pH drops lower than the desired value the diffusion will be reduced and the pH will rise; conversely, if the pH becomes too high then the diffusion rate would increase and the pH would drop. These operating points would be stable for one growth rate and if the growth rate is higher the pressure may be raised to match the new growth rate, according to embodiments of the present invention.

A permeable membrane (integrated into the film bag) may be used to achieve stable pH without the use of buffers, according to embodiments of the present invention. Increasing pressure in the membrane tube provides more carbon to the media for growth, and a reduction in pressure lowers the pH if the pH is too high. If a pH sensor is used with a closed loop system to control the pH by increasing or decreasing the pressure in the membrane tube, the required pressure can be used to infer the growth rate, thus eliminating a need for an expensive turbidity meter, according to embodiments of the present invention.

Because the amount of gas transferred through the membrane is a function of the difference in partial pressures, the amount of CO₂ transferred through the membrane into the media may be modulated by adjusting the pressure in the membrane tube. This provides an inexpensive and reliable way to control the delivery of CO₂ or other gases to the media. Additional control can be achieved by selectively turning on and off different membrane tubes to adjust the total surface area of the membrane in active carbon transfer, or by controlling the concentrations of and constituents of the gases in the membrane tubes. The membrane tube may also include a gas mixture, in which the gas mixture includes carbon dioxide. The carbon dioxide delivery through the membrane tube may be controlled by changing the partial pressure of carbon dioxide within the gas mixture, and/or by changing the overall pressure of the gas mixture, and/or by changing the surface area of the membrane tube. These methods of control may be used in various combinations, along with other methods, to achieve various levels of control, according to embodiments of the present invention.

One or more membrane tubes may also be selectively permeable; in other words, a membrane tube may be configured for permeability to carbon dioxide but not to other gases, according to embodiments of the present invention. A membrane tube configured to remove dissolved oxygen from within the media solution may be permeable to oxygen but not other gases, according to embodiments of the present invention. For example, a stack gas or exhaust gas may be introduced into or circulated through a permeable membrane, which permits dissolved oxygen to enter membrane from the media, but which prohibits other gases (e.g. gases that may be toxic to algae or otherwise undesirable) from crossing into the media from within the permeable membrane, according to embodiments of the present invention. As another example, stack gas or exhaust gas may be introduced into or circulated through a permeable membrane within a photobioreactor, which permits carbon dioxide from within the permeable membrane to enter the media from within the membrane, but which prohibits other gases (e.g. gases that may be toxic to algae or otherwise undesirable) from crossing into the media from within the permeable membrane, according to embodiments of the present invention. According to some embodiments of the present invention, the photobioreactor bag itself is at least partially formed of a permeable membrane, is submerged in water, and stack gas and/or other exhaust gases are introduced directly into the water, such that the carbon dioxide equivalent partial pressure difference between the water and the media inside the photobioreactor bag causes the carbon dioxide from the water to cross into the photobioreactor bag.

Depending on the composition of the media used and desired setpoint for the pH of the media, the pH of the media may be increased by sparging air or other gases through it, according to embodiments of the present invention. This permits an inexpensive and robust way to raise the pH, according to embodiments of the present invention. Similarly, the pH can be reduced by sparging with a higher concentration of CO₂ than is normally found in air. Based on the disclosure provided herein, one of ordinary skill in the art will appreciate the numerous compositions of sparging gas, and the various combinations of gas selection, membrane selection, membrane surface area, and other factors that can be used to affect the pH and/or the algae growth rate. According to some embodiments of the present invention, the membrane tube can be deflated and/or inflated over time to promote mixing.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A photobioreactor comprising:
a flexible outer bag, the flexible outer bag comprising a plastic film;
a media solution contained in the flexible outer bag; and
a membrane tube situated inside of the flexible outer bag, wherein the membrane tube contains carbon dioxide and wherein the membrane tube is gas permeable and is configured to transfer the carbon dioxide from within the membrane tube into the media solution.

2. The photobioreactor of claim 1, wherein the membrane tube is a porous membrane tube.

3. The photobioreactor of claim 1, wherein the membrane tube is a non-porous membrane tube.

4. The photobioreactor of claim 1, wherein the membrane tube is at least partially integral with the flexible outer bag.

5. The photobioreactor of claim 1, wherein the membrane tube extends substantially along a length of the flexible outer bag.

6. The photobioreactor of claim 1, wherein the membrane tube is further configured to permit transfer of dissolved oxygen from the media solution into the membrane tube.

7. The photobioreactor of claim 1, further comprising a sparging tube within the flexible outer bag.

8. The photobioreactor of claim 7, wherein membrane tube is located above the sparging tube within the flexible outer bag.

9. The photobioreactor of claim 1, wherein the membrane tube is molded into the flexible outer bag.

10. The photobioreactor of claim 9, wherein the membrane tube increases durability of the flexible outer bag.

11. The photobioreactor of claim 1, wherein the membrane tube is a first membrane tube, the photobioreactor further comprising a second membrane tube, wherein a first pressure of a first gas within the first membrane tube is controlled independently from a second pressure of a second gas within the second membrane tube.

12. The photobioreactor of claim 1, wherein the membrane tube comprises a single carbon dioxide port in fluid communication with a carbon dioxide source.

13. The photobioreactor of claim 1, wherein the membrane tube comprises a first carbon dioxide port and a second carbon dioxide port, wherein the first carbon dioxide port is in fluid communication with a carbon dioxide source and wherein the second carbon dioxide port is an exhaust port.

14. The photobioreactor of claim 13, further comprising a carbon dioxide recirculation line connecting the first and second carbon dioxide ports.

15. The photobioreactor of claim 13, further comprising an actuator configured to vary a flow rate of carbon dioxide from the first carbon dioxide port to the second carbon dioxide port through the membrane tube.

16. The photobioreactor of claim 15, wherein the actuator includes a configuration that permits the flow rate to be zero.

17. The photobioreactor of claim 1, wherein the membrane tube comprises a single set of one or more carbon dioxide ports in fluid communication with a carbon dioxide source.

18. The photobioreactor of claim 17, wherein the membrane tube lacks an exhaust port.

19. The photobioreactor of claim 1, wherein the media solution comprises algae.

20. The photobioreactor of claim 1, further comprising a liquid bath in which the flexible outer bag is submerged.

21. The photobioreactor of claim 1, wherein a net flow rate of carbon dioxide from the membrane tube into the media solution depends on a carbon dioxide differential between a gas mixture within the membrane tube and the media solution, and wherein adjusting the net flow rate comprises adjusting a partial pressure of carbon dioxide within the gas mixture.

22. The photobioreactor of claim 1, wherein a net flow rate of carbon dioxide from the membrane tube into the media solution depends on a carbon dioxide pressure differential between a gas mixture within the membrane tube and the media solution, and wherein adjusting the net flow rate comprises adjusting the gas mixture pressure in the membrane tube.

23. The photobioreactor of claim 1, wherein a net flow rate of carbon dioxide from the membrane tube into the media solution depends on a permeable membrane effective surface area, and wherein adjusting the net flow rate comprises adjusting the permeable membrane effective surface area.

24. A photobioreactor comprising:
a flexible outer bag, the flexible outer bag comprising a plastic film;
a media solution contained in the flexible outer bag, the media solution holding a photosynthetic organism in suspension; and
a membrane tube situated inside of the flexible outer bag, wherein the membrane tube contains a gas and wherein the membrane tube is gas permeable and is configured to transfer the gas from within the membrane tube into the media solution.

25. A method for gas transfer in a photobioreactor, the method comprising:
filling a flexible outer bag with a media solution, the flexible outer bag comprising a plastic film, the flexible outer bag further comprising a membrane tube inside of the flexible outer bag, wherein the membrane tube is gas permeable and is not liquid permeable; and
pressurizing the membrane tube with carbon dioxide, thereby transferring the carbon dioxide from within the membrane tube into the media solution.

26. The method of claim 25, wherein at least a first portion of the flexible outer bag comprises a membrane that is gas permeable, and wherein a second portion of the flexible outer bag is more transparent than the first portion.

27. The method of claim 25, wherein pressurizing the membrane tube with carbon dioxide comprises pressurizing the membrane tube with a gas composition including carbon dioxide, wherein the gas composition is selected to cause carbon dioxide to enter the media solution from the membrane tube and to cause oxygen to enter the membrane tube from the media solution.

28. The method of claim 25, wherein the membrane tube is a first membrane tube, the method further comprising:
inserting a second membrane tube inside of the flexible outer bag, wherein the second membrane tube is gas permeable and is not liquid permeable; and
pressurizing the second membrane tube with an oxygen stripping gas, wherein dissolved oxygen from the media solution passes through the second membrane tube and into the oxygen stripping gas.

29. The method of claim 25, further comprising submerging the flexible outer bag in a liquid.

30. A photobioreactor comprising:
a flexible outer bag;
a liquid media contained in the flexible outer bag, the liquid media suitable for growing algae, the flexible outer bag comprising:
a first portion comprising plastic film, and
a second portion comprising a gas permeable membrane, wherein the gas permeable membrane is configured to permit dissolved oxygen to transfer from within the liquid media, across the gas permeable membrane, and to an outside of the flexible outer bag; and
a membrane tube situated inside of the flexible outer bag, wherein the membrane tube contains carbon dioxide, wherein the membrane tube is gas permeable and is configured to transfer the carbon dioxide from within the membrane tube into the media solution.

31. A photobioreactor comprising:
a flexible outer bag submerged in a liquid bath; and
a liquid media contained in the flexible outer bag, the liquid media suitable for growing algae, the flexible outer bag comprising:
a first portion comprising plastic film, and
a second portion comprising a gas permeable membrane, wherein the gas permeable membrane is configured to permit dissolved oxygen to transfer from within the liquid media, across the gas permeable membrane, and into the liquid bath, and wherein the gas permeable membrane is configured to permit carbon dioxide to transfer from the liquid bath, across the gas permeable membrane, and into the liquid media.

32. The photobioreactor of claim 31, further comprising:
a membrane tube situated inside of the flexible outer bag, wherein the membrane tube contains carbon dioxide, wherein the membrane tube is gas permeable and is configured to transfer the carbon dioxide from within the membrane tube into the media solution.

33. The photobioreactor of claim 31, wherein the first portion is more transparent than the second portion.

34. A method for making a gas permeable membrane tube, the method comprising:
arranging a first layer next to a second layer, a third layer next to the second layer, and a fourth layer next to the third layer, wherein the second and third layers are each a gas permeable membrane, wherein the first and fourth layers are welding layers, and wherein the first layer includes one or more membrane windows; and
fusing the first, second, third, and fourth layers together around the one or more membrane windows.

35. The method of claim 34, wherein the first layer is formed in a shape corresponding generally to a welding pattern between the first, second, third, and fourth layers, and wherein fusing the first, second, third, and fourth layers comprises fusing the first, second, third, and fourth layers according to the welding pattern.

36. The method of claim 34, further comprising refraining from welding within the one or more membrane windows, in order to minimize damage to the second and third layers.

37. The method of claim 34, wherein each of the first and fourth layers comprise a composite film that includes a polyethylene layer, a nylon layer, and one or more tie layers between the polyethylene layer and the nylon layer.
